(19) United States
(12) Patent Application Publication
(10) Pub. No.: US 2005/0075537 A1
(43) Pub. Date: Apr. 7, 2005

(54) Method and system for real-time automatic abnormality detection for in vivo images

(75) Inventors: Shoupu Chen, Rochester, NY (US); Lawrence A. Ray, Rochester, NY (US); Nathan D. Cahill, West Henrietta, NY (US); Marvin M. Goodgame, Ontario, NY (US)

Correspondence Address: Thomas H. Close;Patent Legal Staff, Eastman Kodak Company, 343 State Street, Rochester, NY, 14650-2201, US

(73) Assignee: Eastman Kodak Company

(21) Appl. No.: 10/679,711

(22) Filed: Oct. 6, 2003

(51) Int. Cl. A61B 6/00; A61B 1/04

(52) U.S. Cl. 600/109; 600/476

(57) ABSTRACT

A digital image processing method for real-time automatic abnormality detection of in vivo images, comprising the steps of: acquiring images using an in vivo video camera system; forming an in vivo video camera system examination bundlette; transmitting the examination bundlette to proximal in vitro computing device(s); processing the transmitted examination bundlette; automatically identifying abnormalities in the transmitted examination bundlette; and setting off alarming signals to a local site provided that suspected abnormalities have been identified.

### FIELD OF THE INVENTION

The present invention relates generally to an endoscopic imaging system and, in particular, to real-time automatic abnormality detection of in vivo images.

### BACKGROUND OF THE INVENTION

Several in vivo measurement systems are known in the art. They include swallowed electronic capsules which collect data and which transmit the data to an external receiver system. These capsules, which are moved through the digestive system by the action of peristalsis, are used to measure pH (“Heidelberg” capsules), temperature (“CoreTemp” capsules), and pressure throughout the gastro-intestinal (GI) tract. They have also been used to measure gastric residence time, which is the time it takes for food to pass through the stomach and intestines. These capsules typically include a measuring system and a transmission system, wherein the measured data is transmitted at radio frequencies to a receiver system.

U.S. Pat. No. 5,604,531, issued Feb. 18, 1997 to Iddan et al., titled “IN VIVO VIDEO CAMERA SYSTEM” teaches an in vivo measurement system, in particular an in vivo camera system, which is carried by a swallowed capsule. In addition to the camera system there is an optical system for imaging an area of the GI tract onto the imager and a transmitter for transmitting the video output of the camera system. The overall system, including a capsule that can pass through the entire digestive tract, operates as an autonomous video endoscope. It images even the difficult to reach areas of the small intestine.

U.S. patent application Ser. No. 2003/0023150 A1, filed Jul. 25, 2002 by Yokoi et al., titled “CAPSULE-TYPE MEDICAL DEVICE AND MEDCAL SYSTEM” teaches a swallowed capsule-type medical device which is advanced through the inside of the somatic cavities and lumens of human beings or animals for conducting examination, therapy, or treatment. Signals including images captured by the capsule-type medical device are transmitted to an external receiver and recorded on a recording unit. The images recorded are retrieved in a retrieving unit and displayed on the liquid crystal monitor and to be compared by an endoscopic examination crew with past endoscopic disease images that are stored in a disease image database.

The examination requires the capsule to travel through the GI tract of an individual, which will usually take a period of many hours. A feature of the capsule is that the patient need not be directly attached or tethered to a machine and may move about during the examination. While the capsule will take several hours to pass through the patient, images will be recorded and will be available while the examination is in progress. Consequently, it is not necessary to complete the examination prior to analyzing the images for diagnostic purposes. However, it is unlikely that trained personnel will monitor each image as it is received. This process is too costly and inefficient. However, the same images and associated information can be analyzed in a computer-assisted manner to identify when regions of interest or conditions of interest present themselves to the capsule. When such events occur, then trained personnel will be alerted and images taken slightly before the point of the alarm and for a period thereafter can be given closer scrutiny. Another advantage of this system is that trained personnel are alerted to an event or condition that warrants their attention. Until such an alert is made, the personnel are able to address other tasks, perhaps unrelated to the patient of immediate interest.

Using computers to examine and to assist in the detection from images is well known. Also, the use of computers to recognize objects and patterns is also well known in the art. Typically, these systems build a recognition capability by training on a large number of examples. The computational requirements for such systems are within the capability of commonly available desk-top computers. Also, the use of wireless communications for personal computers is common and does not require excessively large or heavy equipment. Transmitting an image from a device attached to the belt of the patient is well-known.

Notice that 0023150 teaches a method of storing the in vivo images first and retrieving them later for visual inspection of abnormalities. The method lacks of abilities of prompt and real-time automatic detection of abnormalities, which is important for calling for physicians' immediate attentions and actions including possible adjustment of the in vivo imaging system's functionality. Notice also that, in general, using this type of capsule device, one round of imaging could produce thousands and thousands of images to be stored and visually inspected by the medical professionals. Obviously, the inspection method taught by 0023150 is far from efficient.

WO Patent Application No. 02/073507 A2, filed Mar. 14, 2002 by Doron Adler et al., titled “METHOD AND SYSTEM FOR DETECTING COLORIMETRIC ABNORMALITIES,” and incorporated herein by reference, teaches a method for detecting colorimetric abnormalities using a swallowed capsule-type medical device which is advanced through the inside of the somatic cavities and lumens. The taught method is limited to the scope of constructing an algorithm and a system that is capable of detecting only one of a plurality of possible GI tract abnormalities (in this case, color) as opposed to other GI tract abnormalities such as texture, shape, and other physical measures. Moreover, WO Application No. 02/073507 teaches a method to detect calorimetric abnormalities for a patient using an image monitor viewed by a physician, which is too costly and inefficient. WO Application No. 02/073507 teaches a method lacking of systematically using information, other than image data, such as patient's metadata (to be defined later), for automatic abnormality detection, recording, and retrieving.

It is useful to design an endoscopic in vivo imaging system that is capable of detecting an abnormality in real-time. (Herein, throughout this patent application, ‘real-time’ means that the abnormality detection process starts as soon as an in vivo image becomes available while the capsule containing the imaging system is traveling throughout the body. There is no need to wait for the imaging system within the capsule to finish its imaging of the whole GI tract. Such ‘real-time’ imaging is different than capturing images in very short periods of time). Additionally an in vivo imaging system will also be useful in automatically detecting, recording, and retrieving images of GI tract abnormalities.

There is a need therefore for an improved endoscopic imaging system that overcomes the problems set forth above and addresses the utilitarian needs set forth above.

These and other aspects, objects, features and advantages of the present invention will be more clearly understood and appreciated from a review of the following detailed description of the embodiments and appended claims, and by reference to the accompanying drawings.

### SUMMARY OF THE INVENTION

The need is met according to the present invention by providing a digital image processing method for real-time automatic abnormality detection of in vivo images that includes forming an examination bundlette of a patient that includes real-time captured in vivo images; processing the examination bundlette; automatically detecting one or more abnormalities in the examination bundlette based on predetermined criteria for the patient; and signaling an alarm provided that the one or more abnormalities in the examination bundlette have been detected.

### BRIEF DESCRIPTION OF THE DRAWINGS

FIG. 1 is a prior art block diagram illustration of an in vivo camera system;

FIG. 2A is an illustration of the concept of an examination bundle of the present invention;

FIG. 2B is an illustration of the concept of an examination bundlette of the present invention;

FIG. 3 is a flowchart illustrating information flow of the real-time abnormality detection method of the present invention;

FIG. 4 is a schematic diagram of an examination bundlette processing hardware system useful in practicing the present invention;

FIG. 5 is a flowchart illustrating abnormality detection of the present invention;

FIG. 6 is a flowchart illustrating image feature examination of the present invention;

FIGS. 7a and 7b are one dimensional and two dimensional graphs, respectively, illustrating thresholding operations;

FIGS. 8a, 8B, 8C, and 8D are illustrations of four images related to in vivo image abnormality detection of the present invention;

FIG. 9 is a flowchart illustrating color feature detection of the present invention;

FIGS. 10A and 10B are illustrations of two graphs of generalized RG space of the present invention; and

FIG. 11 is an illustration of a data collection device.

To facilitate understanding, identical reference numerals have been used, where possible, to designate identical elements that are common to the figures.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well-known features may be omitted or simplified in order not to obscure the present invention.

During a typical examination of a body lumen, a conventional in vivo camera system captures a large number of images. The images can be analyzed individually, or sequentially, as frames of a video sequence. An individual image or frame without context has limited value. Some contextual information is frequently available prior to or during the image collection process; other contextual information can be gathered or generated as the images are processed after data collection. Any contextual information will be referred to as metadata. Metadata is analogous to the image header data that accompanies many digital image files.

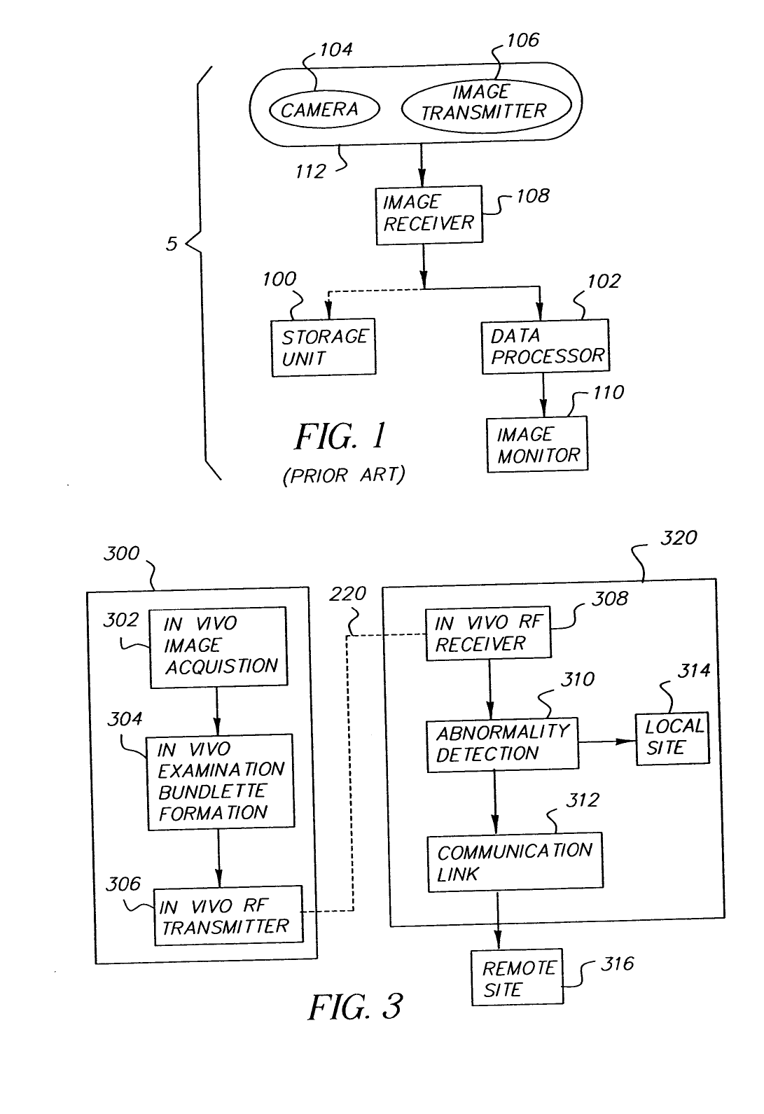

FIG. 1 shows a prior art block diagram of the in vivo video camera system 5 described in U.S. Pat. No. 5,604,531. The in vivo video camera system 5 captures and transmits images of the GI tract while passing through the gastro-intestinal lumen. The in vivo video camera system 5 includes a storage unit 100, a data processor 102, a camera 104, an image transmitter 106, an image receiver 108 which usually includes an antenna array, and an image monitor 110. Storage unit 100, data processor 102, image monitor 110, and image receiver 108 are located outside the patient's body. Camera 104, as it transits the GI tract, is in communication with image transmitter 106 located in capsule 112 and image receiver 108 located outside the body. Data processor 102 transfers frame data to and from storage unit 100 while the former analyzes the data. Processor 102 also transmits the analyzed data to image monitor 110 where a physician views it. The data can be viewed in real-time or at some later date. Here, throughout this patent application, ‘real-time’ means that the abnormality detection process starts as soon as an in vivo image becomes available while the capsule 112 containing the imaging system is traveling throughout the body. There is no need to wait for the imaging system within the capsule to finish its imaging of the whole GI tract. Such ‘real-time’ imaging is different than capturing images in very short periods of time.

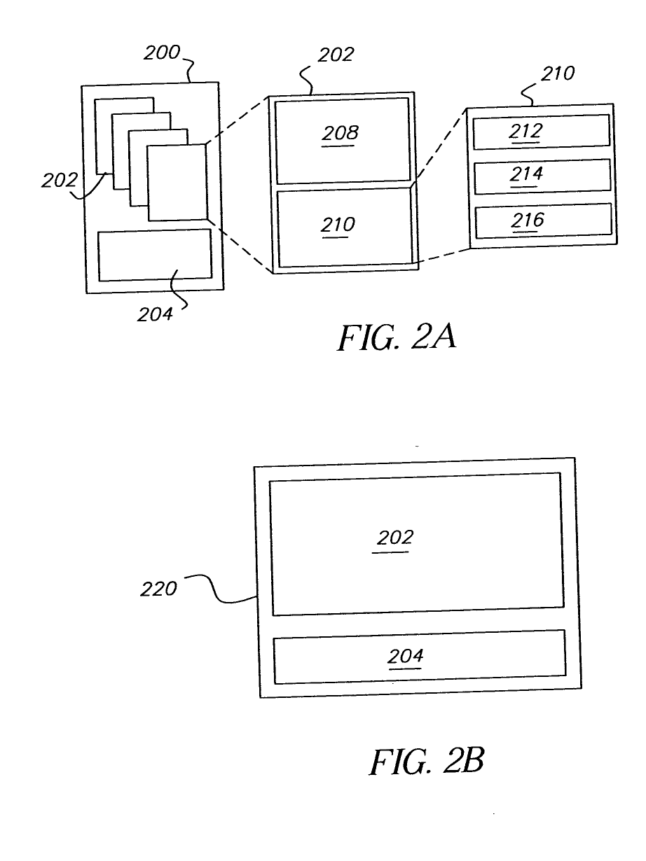

Referring to FIG. 2A, the complete set of all images captured during the examination, along with any corresponding metadata, will be referred to as an examination bundle 200. The examination bundle 200 consists of a plurality of individual image packets 202 and a section containing general metadata 204.

An image packet 202 comprises two sections: the pixel data 208 of an image that has been captured by the in vivo camera system, and image specific metadata 210. The image specific metadata 210 can be further refined into image specific collection data 212, image specific physical data 214, and inferred image specific data 216. Image specific collection data 212 includes information such as the frame index number, frame capture rate, frame capture time, and frame exposure level. Image specific physical data 214 includes information such as the relative position of the capsule 112 when the image was captured, the distance raveled from the position of initial image capture, the instantaneous velocity of the capsule 112, capsule orientation, and non-image sensed characteristics such as pH, pressure, temperature, and impedance. Inferred image specific data 216 includes location and description of detected abnormalities within the image, and any pathologies that have been identified. This data can be obtained either from a physician or by automated methods.

The general metadata 204 includes such information as the date of the examination, the patient identification, the name or identification of the referring physician, the purpose of the examination, suspected abnormalities and/or detection, and any information pertinent to the examination bundle 200. The general metadata 204 can also include general image information such as image storage format (e.g., TIFF or JPEG), number of lines, and number of pixels per line.

Referring to FIG. 2B, a single image packet 202 and the general metadata 204 are combined to form an examination bundlette 220 suitable for real-time abnormality detection. The examination bundlette 220 differs from the examination bundle 200 in that the examination bundle 200 requires the GI tract to be imaged completely during travel of the capsule 112. In contrast, the examination bundlette 220 requires only a portion of the GI tract to be imaged as corresponding to the real-time imaging disclosed herein.

It will be understood and appreciated that the order and specific contents of the general metadata or image specific metadata may vary without changing the functionality of the examination bundle 200.

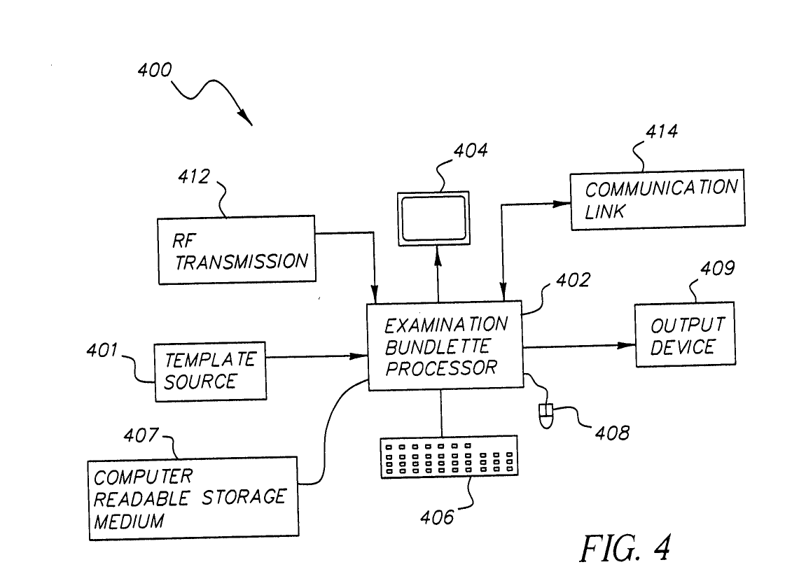

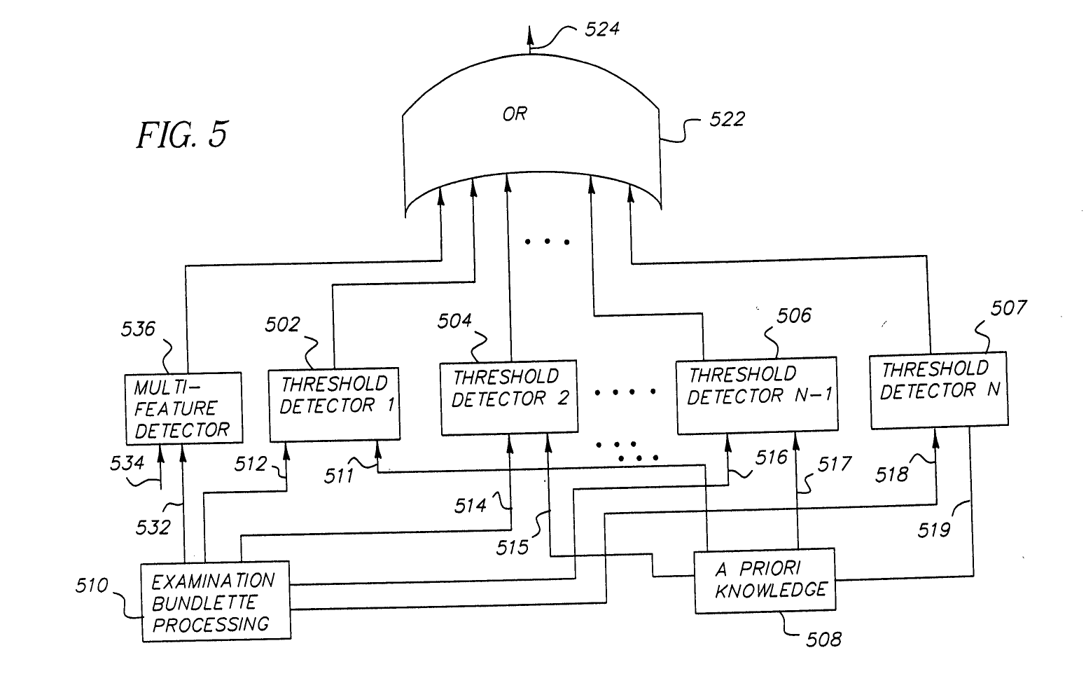

Referring now to FIGS. 2A and 3, an exemplary embodiment of the present invention is described. FIG. 3 is a flowchart illustrating the real-time automatic abnormality detection method of the present invention. In FIG. 3, an in vivo imaging system 300 can be realized by using systems such as the swallowed capsule described in U.S. Pat. No. 5,604,531 for the present invention. An in vivo image 208, shown in FIG. 2A, is captured in an in vivo image acquisition step 302. During In Vivo Examination Bundlette Formation step 304, the image 208 is combined with image specific metadata 210 to form an image packet 202, as shown in FIG. 2. The image packet 202 is further combined with general metadata 204 and compressed to become an examination bundlette 220. The examination bundlette 220 is transmitted, through radio frequency, to a proximal in vitro computing device in RF transmission step 306. An in vitro computing device 320 is either a portable computer system attached to a belt worn by the patient or in near proximity to a patient. Alternatively, it is a system such as shown in FIG. 4 and will be described in detail later. The transmitted examination bundlette 220 is received in the proximal in vitro computing device 320 during an In Vivo RF Receiver step 308. Data received in the in vitro computing device 320 is examined for any sign of disease in an Abnormality detection step 310. The step of Abnormality detection 310 is further detailed in FIG. 5

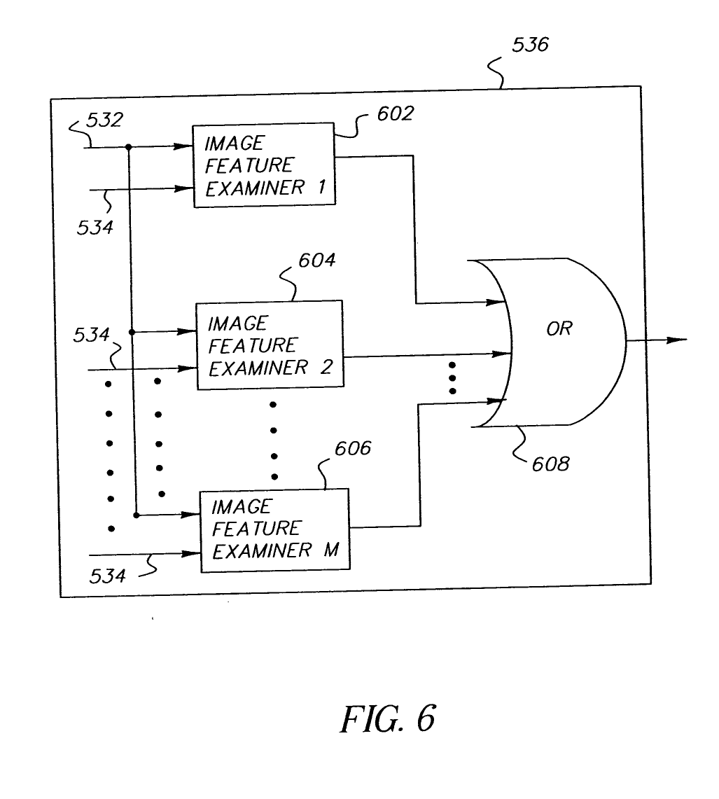

Referring to FIG. 5, the examination bundlette 220 is first decompressed, decomposed, and processed in the examination bundlette processing step 510. During the examination bundlette step 510, the image data portion of the examination bundlette 220 is subjected to image processing algorithms such as filtering, enhancing, and geometric correction. These algorithms can be implemented in color space or grayscale space. There are a plurality of threshold detectors, 502, 504, 506, and 507, each capable of handling one of the non-image sensed characteristics in the GI tract such as pH 512, pressure 514, temperature 516, and impedance 518. Distributions and thresholds of the non-image sensed characteristics such as pH 512, pressure 514, temperature 516, and impedance 518 are learned in a step of a priori knowledge 508. If values of the non-image sensed characteristics such as pH 512, pressure 514, temperature 516, and impedance 518 pass over their respective thresholds 511, 515, 517, and 519, corresponding alarm signals are sent to a logic OR gate 522. Also in FIG. 5, there is a Multi-feature Detector 536 which is detailed in FIG. 6.

Referring to FIG. 6, there is a plurality of image feature detectors, each of which examines one of the image features of interest. Image features such as color, texture, and geometric shape of segmented regions of the GI tract image 532 are extracted and automatically compared to predetermined templates 534 by one of the image feature examiners 602, 604, or 606. The predetermined templates 534 are statistical representations of GI image abnormality features through supervised learning. If any one of the multi-features in image 532 matches its corresponding template or within the ranges specified by the templates, an OR gate 608 sends an alarm signal to the OR gate 522, shown in FIG. 5.

Referring to FIGS. 5 and 3, any combination of the alarm signals from detectors 536, 502, 504, 506, and 507 will prompt the OR gate 522 to send a signal 524 to a local site 314 and to a remote health care site 316 through communication link 312. An exemplary communication link 312 could be a broadband network connected to the in vitro computing system 320. The connection from the broadband network to the in vitro computing system 320 could be either a wired connection or a wireless connection.

An exemplary image feature detection is the color detection for Hereditary Hemorrhagic Telangiectasia disease. Hereditary Hemorrhagic Telangiectasia (HHT), or Osler-Weber-Rendu Syndrome, is not a disorder of blood clotting or missing clotting factors within the blood (like hemophilia), but instead is a disorder of the small and medium sized arteries of the body. HHT primarily affects 4 organ systems; the lungs, brain, nose, and gastrointestinal (stomach, intestines, or bowel) system. The affected arteries either have an abnormal structure causing increased thinness or an abnormal direct connection with veins (arteriovenous malformation). Gastrointestinal tract (stomach, intestines, or bowel) bleeding occurs in approximately 20 to 40% of persons with HHT. Telangiectasias often appear as bright red spots in the gastrointestinal tract.

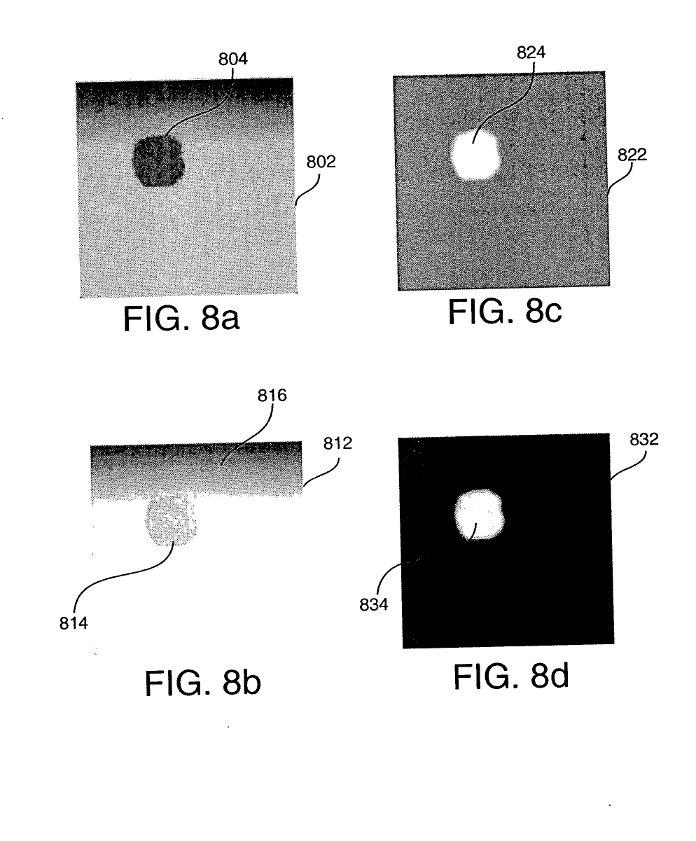

A simulated image of a telangiectasia 804 on a gastric fold is shown in image 802 in FIG. 8A. Note that the color image 802 is shown in FIG. 8A as a gray scale (black and white) image. To human eyes, the red component of the image provides distinct information for identifying the telangiectasia 804 on the gastric fold. However, for the automatic telangiectasia detection using a computer, the native red component alone as shown by red image 812 of the color image 802, in fact, is not able to clearly distinguish the foreground (telangiectasia 814) and the part of the background 816 of image 812 in terms of pixel intensity values.

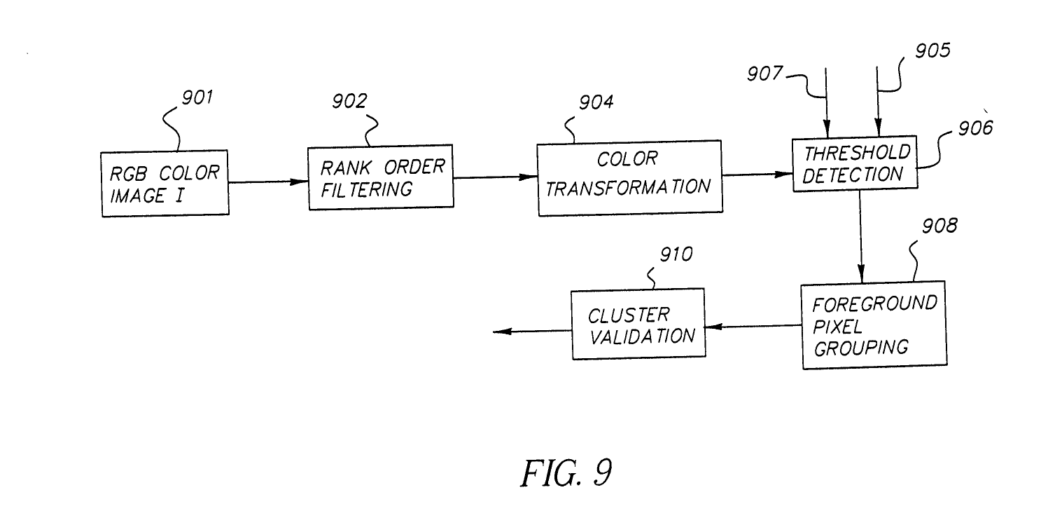

To solve the problem, the present invention devises a color feature detection algorithm that detects the telangiectasia 804 automatically in an in vivo image. Referring to FIG. 9, the color feature detection performed according to the present invention by the multi-feature detector 536, shown in FIG. 5, will be described. The color digital image 901, expressed in a device independent RGB color space is first filtered in a rank order filtering step 902. One exemplary rank order filtering is median filtering. Denote the input RGB image by I_{RGB}={Cᵢ}, where i=1, 2, 3 for R, G, and B color planes, respectively. A pixels at location (m, n) in a plane Cᵢ is represented by pᵢ(m, n), where m=0, . . . M−1 and n=0, . . . N−1, M is the number of rows, and N is the number of columns in a plane. Exemplary values for M and N are 512 and 768. The median filtering is defined as

$${p}_{i} ( m , n ) = \{ \begin{matrix}median ( {C}_{i} , m , n , S , T ) {\vert}_{mediun ( {C}_{i} , m , n , S , T ) > {T}_{Low}} \\ 0 {\vert}_{otherwise } \end{matrix} \qquad ( Equation 1 )$$

where T_{Low} is a predefined threshold. An exemplary value for T_{Low} is 20. S and T are the width and height of the median operation window. Exemplary values for S and T are 3 and 3. This operation is similar to the traditional process of trimmed median filtering well known to people skilled in the art. Notice that the purpose of the median filtering in the present invention is not to improve the visual quality of the input image as traditional image processing does; rather, it is to reduce the influence of a patch or patches of pixels that have very low intensity values at the threshold detection stage 906. A patch of low intensity pixels is usually caused by a limited illumination power and a limited viewing distance of the in vivo imaging system as it travels down to an opening of an organ in the GI tract. This median filtering operation also effectively reduces noises.

In color transformation step 904, after the media filtering, I_{RGB} is converted to a generalized RGB image, I_{gRGB} using the formula:

$${\overline{p}}_{j} ( m , n ) = \frac{{p}_{j} ( m , n )}{{\sum }_{i}^{} {p}_{i} ( m , n )} \qquad ( Equation 2 )$$

where pᵢ(m, n) is a pixel of an individual image plane i of the median filtered image I_{RGB}. \overline{p}ᵢ(m, n) is a pixel of an individual image plane i of the resultant image I_{gRGB}. This operation is not valid when

$${\sum }_{i} {p}_{i} ( m , n ) = 0 ,$$

and the output, \overline{p}ᵢ(m, n), will be set to zero. The resultant three new elements are linearly dependent, that is,

$${\sum }_{j} {\overline{p}}_{j} ( m , n ) = 0 ,$$

so that only two elements are needed to effectively form a new space that is collapsed from three dimensions to two dimensions. In most cases, \overline{p}₁ and \overline{p}₂, that is, generalized R and G, are used. In the present invention, to detect a telangiectasia 804, the converted generalized R component is needed. FIG. 8C displays the converted generalized R component of the image depicted in FIG. 8A. Clearly, pixels in region 824 have distinguishable values comparing to pixels in the background region. Therefore, a simple thresholding operation 906 can separate the pixels in the foreground (i.e., telangiectasia 824) from the background.

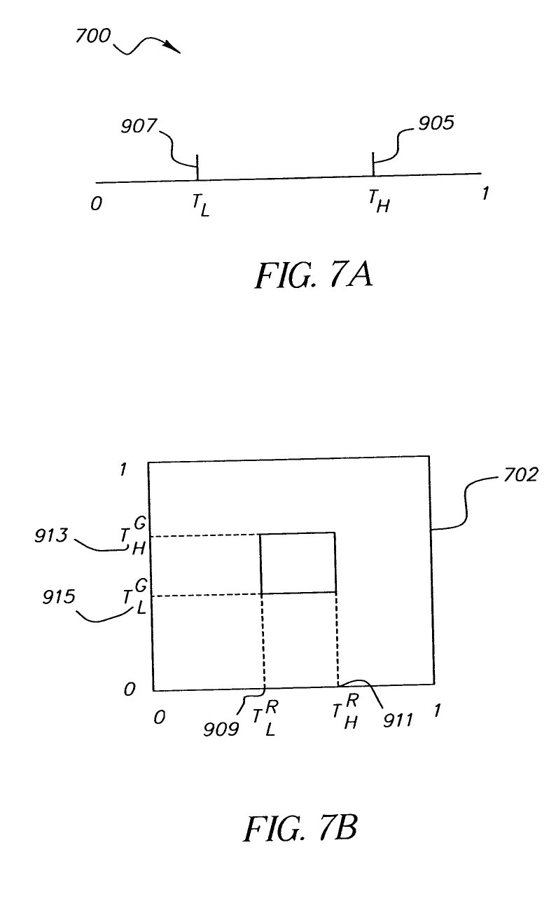

It is not a trivial task to parameterize the sub-regions of thresholding color in (R, G, B) space. With the help of color transformation 904, the generalized R color is identified to be the parameter to separate a disease region from a normal region. Referring to FIG. 7A, a one-dimensional graph 700 of the generalized R color of disease region pixels and the normal region pixels based on a histogram analysis provides useful information for partitioning the disease region pixels and the normal region pixels. The histogram is a result of a supervised learning of sample disease pixels and normal pixels in the generalized R space. A measured upper threshold parameter T_{H} 905 (part of 534) and a measured lower threshold parameter T_{L} 907 (part of 534) obtained from the histogram are used to determine if an element \overline{p}₁(m, n) is a disease region pixel (foreground pixel) or a normal region pixel:

$$b ( m , n ) = \{ \begin{matrix}1 & if {T}_{L} < {\overline{p}}_{1} ( m , n ) < {T}_{H} \\ 0 & else \end{matrix} \qquad ( Equation 3 )$$

where b(m, n) is an element of a binary image I_{Binary} that has the same size as I_{gRGB}. Exemplary value for T_{L} is 0.55, and exemplary value for T_{H} is 0.70. Thus, FIG. 7A illustrates the thresholding operation range.

Referring to FIG. 8D in conjunction with FIG. 9, FIG. 8D is an exemplary binary image I_{Binary} of the image in FIG. 8A after the thresholding operation 906. Pixels having value 1 in the binary image I_{Binary} are the foreground pixels. Foreground pixels are grouped in foreground pixel grouping step 908 to form clusters such as cluster 834. A cluster is a non-empty set of 1-valued pixels with the property that any pixel within the cluster is also within a predefined distance to another pixel in the cluster. Step 908 groups binary pixels into clusters based upon this definition of a cluster. However, it will be understood that pixels may be clustered on the basis of other criteria.

Under certain circumstances, a cluster of pixels may not be valid. Accordingly, a step of validating the clusters is needed. It is shown in FIG. 9 as Cluster Validation step 910. A cluster may be invalid if it contains too few binary pixels to acceptably determine the presence of an abnormality. For example, if the number of pixels in a cluster is less than V, then this cluster is invalid. Example V value could be 3. If there exist one or more valid clusters, an alarm signal will be generated and sent to OR gate 608, shown in FIG. 6. This alarm signal is also saved to the examination bundlette 220 for record.

Note that in Equation 1, pixels, pᵢ(m, n), having value less than T_{Low} are excluded from the detection of abnormality. A further explanation of the exclusion is given below for conditions other than the facts stated previously.

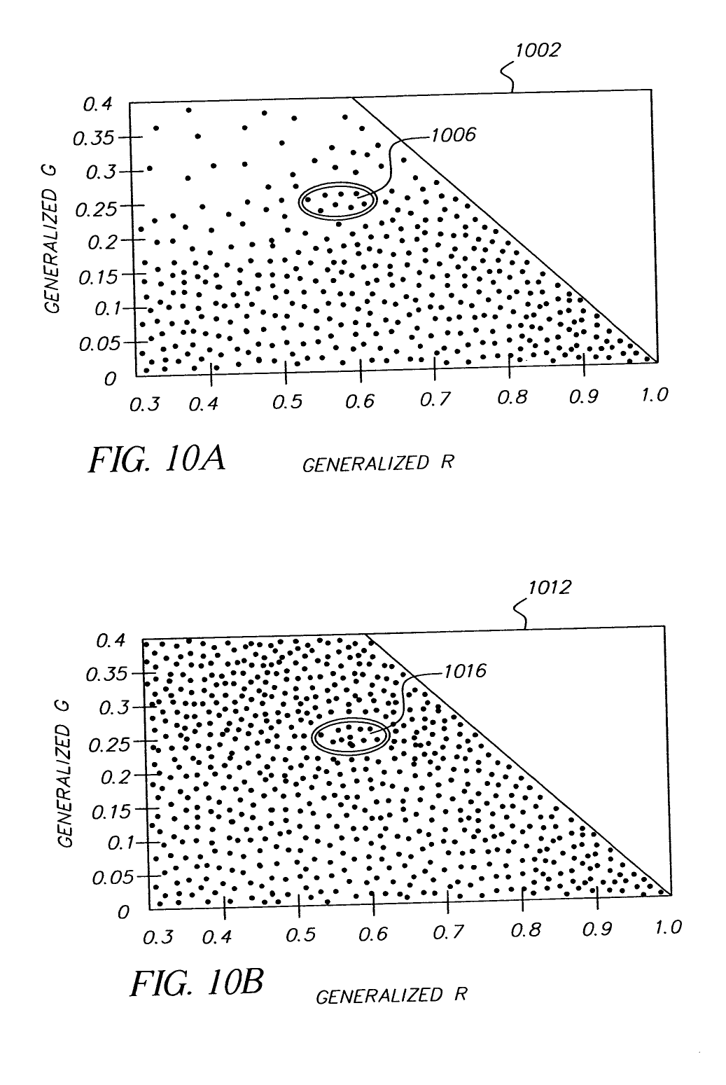

Referring to FIGS. 10A and 10B, there are two graphs 1002 and 1012, respectively, showing a portion of the generalized RG space. At every point in the generalized RG space, a corresponding color in the original RGB space fills in. In fact, the filling of original RGB color in the generalized RG space is a mapping from the generalized RG space to the original RGB space. This is not a one-to-one mapping. Rather, it is a one-to-many mapping. Meaning that there could be more than one RGB colors that are transformed to a same point in the generalized space. Graphs 1002 and 1012 represent two of a plurality of possible mappings from the generalized RG space to the original RGB space. Now in relation to the abnormality detection problem, region 1006 in graph 1002 indicates the generalized R and G values for a disease spot in the gastric fold, and region 1016 in graph 1012 does the same. Region 1006 maps to colors belonging to a disease spot in the gastric fold in a normal illumination condition. On the other hand, region 1016 maps to colors belonging to places having low reflection in a normal illumination condition. Pixels having these colors mapped from region 1016 are excluded from further consideration to avoid frequent false alarms.

Also note that for more robust abnormality detection, as an alternative, threshold detection 906, in FIG. 9, can use both generalized R and G to further reduce false positives. In this case and referring to a two-dimensional graph 702 shown in FIG. 7B, the upper threshold parameter T_{H} 905 (shown in FIG. 7A) is a two-dimensional array containing T_{H} ^{G} 913 and T_{H}^{R} 911 for generalized G and R respectively. Exemplary values are 0.28 for T_{H}^{G} and 0.70 for T_{H}^{R}. At the same time, the lower threshold parameter T_{L} 907 (shown in FIG. 7A) is also a two-dimensional array containing T_{L}^{G} 915 and T_{L}^{R} 909 for generalized G and R respectively. Exemplary values are 0.21 for T_{L}^{G}, and 0.55 for T_{L}^{R}. In a transformed in vivo image I_{gRGB}, if the elements \overline{p}₁(m, n) and \overline{p}₂(m, n) of a pixel are between the range of T_{L}^{R} and T_{H}^{R} and the range of T_{L} ^{G} and T_{H}^{G}, then the corresponding pixel b(m, n) of the binary image I_{Binary} is set to one. Thus, FIG. 7B illustrates thresholding ranges for this operation.

Referring again to FIG. 4, illustrated is an exemplary embodiment of an examination bundlette processing hardware system 400 useful in practicing the present invention including a template source 401 and an RF receiver 412. The template from the template source 401 is provided to an examination bundlette processor 402, such as a personal computer, or work station such as a Sun Sparc workstation. The RF receiver 412 passes the examination bundlette 220 to the examination bundlette processor 402. The examination bundlette processor 402 preferably is connected to a CRT display 404, an operator interface such as a keyboard 406 and a mouse 408. Examination bundlette processor 402 is also connected to computer readable storage medium 407. The examination bundlette processor 402 transmits processed digital images and metadata to an output device 409. Output device 409 can comprise a hard copy printer, a long-term image storage device, and/or a connection to another processor. The examination bundlette processor 402 is also linked to a communication link 414 or a telecommunication device connected, for example, to a broadband network.

It is well understood that the transmission of data over wireless links is more prone to requiring the retransmission of data packets than wired links. There is a myriad of reasons for this, a primary one in this situation is that the patient moves to a point in the environment where electromagnetic interference occurs. Consequently, it is preferable that all data from the examination bundle 200 be transmitted to a local computer with a wired connection. Such data transmission has additional benefits, such as the processing requirements for image analysis are easily met, and the primary role of the data collection device on the patient's belt is not burdened with image analysis. It is reasonable to consider the system to operate as a standard local area network (LAN).

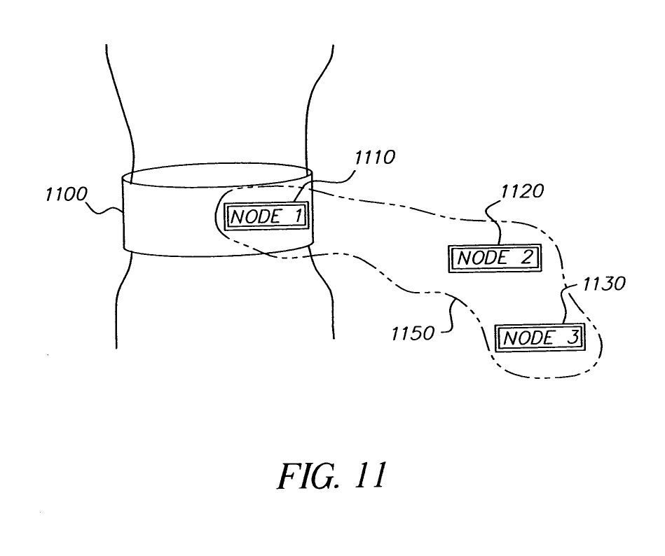

Referring to FIG. 11, a data collection device @node 1 (1110) on a patient's belt 1100 is one node on a LAN 1150. The transmission from the data collection device @node 1 (1110) on the patient's belt 1100 is initially transmitted to a local data collection device @node 2 (1120) or data collection device @node 3 (1130) on the LAN 1150 enabled to communicate with the portable patient belt 1100 and a wired communication network. The wireless communication protocol IEEE-802.11, or one of its successors, is implemented for this application. It is clear that the examination bundle 200 is stored locally within the data collection device @node 1 (1110) on the patient's belt 1100, as well as at a device in wireless contact with the data collection device @node 1 (1110) on the patient's belt 1100. However, it will be appreciated that this is not a requirement for the present invention, only a single operating example. The second data collection device @node 2 (1120) on the LAN 1150 has fewer limitations than the first node at the data collection device @node 1 (1110), as it has a virtually unlimited source of power. Additionally, weight and physical dimensions are not as restrictive as at the data collection device @node 1 (1110) and the first node. Consequently, it is preferable for the image analysis to be conducted on the second data collection device @node 2 (1120) of the LAN 1150. Another advantage of the second data collection device @node 2 (1120) is that it provides a “back-up” of the image data in case some malfunction occurs during the examination. When data collection device @node 2 (1120) detects a condition that requires the attention of trained personnel, then this node system transmits to a remote site where trained personnel are present, a description of the condition identified, the patient identification, identifiers for images in the examination bundle 200, and a sequence of pertinent examination bundlettes 220. The trained personnel can request additional images to be transmitted, or for the image stream to be aborted if the alarm is declared a false alarm.

For people skilled in the art, it is understood that the real-time abnormality detection algorithm of the present invention can be included directly in the design of in vivo imaging capsule on board image processing system.

The invention has been described in detail with particular reference to certain preferred embodiments thereof, but it will be understood that variations and modifications can be effected within the spirit and scope of the invention.

Parts List

5 in vivo video camera system
100 storage unit
102 data processor
104 camera
106 image transmitter
108 image receiver
110 image monitor
112 capsule
200 examination bundle
202 image packets
204 general metadata
208 pixel data
210 image specific metadata
212 image specific collection data
214 image specific physical data
216 inferred image specific data
220 examination bundlette
300 in vivo imaging system
302 in vivo image acquisition
304 forming examination bundlette
306 RF transmission
308 RF receiver
310 abnormality detection
312 communication connection
314 local site
316 remote site
320 in vitro computing device
400 examination bundlette processing hardware system
401 template source
402 examination bundlette processor
404 image display
406 data and command entry device
407 computer readable storage medium
408 data and command control device
409 output device
412 RF transmission
414 communication link
502 threshold detector
504 threshold detector
506 threshold detector
507 threshold detector
508 priori knowledge
510 examination bundlette processing
512 input
514 input
516 input
518 input
511 input
515 input
517 input
519 input
522 OR gate
524 output
532 image
534 templates
536 multi-feature detector
602 image feature examiner
604 image feature examiner
606 image feature examiner
608 OR gate
700 graph of thresholding operation range
702 graph
802 color in vivo image
804 telangiectasia (red spot)
812 R component image
814 spot (foreground)
816 dark area (background)
822 generalized R image
824 spot
832 binary image
834 spot
901 image
902 median filtering
904 color transformation
905 threshold
906 threshold detection
907 threshold
908 foreground pixel grouping
909 lower threshold for generalized R
910 cluster validation
911 upper threshold for generalized G
913 upper threshold for generalized R
915 lower threshold for generalized G
1002 generalized RG space graph
1006 region
1012 generalized RG space graph
1016 region
1100 patient belt
1110 data collection device @node 1
1120 data collection device @node 2.
1130 data collection device @node 3
1150 local area network (LAN)

1. A digital image processing method for real-time automatic abnormality detection of in vivo images, comprising the steps of:
a) forming an examination bundlette of a patient that includes real-time captured in vivo images;
b) processing the examination bundlette;
c) automatically detecting one or more abnormalities in the examination bundlette based on predetermined criteria for the patient; and
d) signaling an alarm provided that the one or more abnormalities in the examination bundlette have been detected.

2. The method claimed in claim 1, wherein the step of forming the examination bundlette, includes the steps of:
a1) forming an image packet of the real-time captured in vivo images of the patient;
a2) forming patient metadata; and
a3) combining the image packet and the patient metadata into the examination bundlette.

3. The method claimed in claim 1, wherein the step of processing the examination bundlette, includes the steps of:
b1) separating the in vivo images from the examination bundlette; and
b2) processing the in vivo images according to selected image processing methods.

4. The method claimed in claim 3, wherein the selected image processing methods include color space conversion and/or noise filtering.

5. The method claimed in claim 4, wherein the color space conversion converts the in vivo images from RGB space to generalized RGB space.

6. The method claimed in claim 1, wherein the step of automatically detecting the one or more abnormalities in the examination bundlette includes the steps of:
c1) detecting parameters that exceed a given threshold of physical data as identified in the in vivo images.

7. The method claimed in claim 1, wherein the step of automatically detecting the one or more abnormalities includes the steps of:
c1) detecting parameters that are substantially different from a given geometric template of physical data as identified in the in vivo images.

8. The method claimed in claim 6, wherein the given threshold is based on statistical data according to the predetermined criteria.

9. The method claimed in claim 7, wherein the geometric template is formed by training a template according to the predetermined criteria.

10. The method claimed in claim 1, wherein the step of signaling the alarm includes the steps of:
d1) providing a communication channel to a remote site; and
d2) sending the alarm to the remote site.

11. The method claimed in claim 1, wherein the step of signaling the alarm includes the steps of:
d1) providing a communication channel to a local site; and
d2) sending the alarm to the local site.

12. A digital image processing system for real-time automatic abnormality detection of in vivo images, comprising:
a) means for forming an examination bundlette of a patient that includes real-time captured in vivo images;
b) means for processing the examination bundlette;
c) means for automatically detecting one or more abnormalities in the examination bundlette based on predetermined criteria for the patient; and
d) means for signaling an alarm provided that the one or more abnormalities in the examination bundlette have been detected.

13. The system claimed in claim 12, wherein the means for forming the examination bundlette, further comprises:
a1) means for forming an image packet of the real-time captured in vivo images of the patient;
a2) means for forming patient metadata; and
a3) means for combining the image packet and the patient metadata into the examination bundlette.

14. The system claimed in claim 12, wherein the means for processing the examination bundlette, further comprises:
b1) means for separating the in vivo images from the examination bundlette; and
b2) means for processing the in vivo images according to selected image processing methods.

15. The system claimed in claim 14, wherein the selected image processing methods include color space conversion and/or noise filtering.

16. The system claimed in claim 15, wherein the color space conversion converts the in vivo images from RGB space to generalized RGB space.

17. The system claimed in claim 12, wherein the means for automatically detecting abnormalities further comprises:
c1) means for detecting parameters that exceed a given threshold of physical data as identified in the in vivo images.

18. The system claimed in claim 12, wherein the means for automatically detecting abnormalities further comprises:
c1) means for detecting parameters that are substantially different from a given geometric template of physical data as identified in the in vivo images.

19. The system claimed in claim 17, wherein the given threshold is based on statistical data according to the predetermined criteria.

20. The system claimed in claim 18, wherein the geometric template is formed by training a template according to the predetermined criteria.

21. The system claimed in claim 12, wherein the means for signaling the alarm further comprises:
d1) means for providing a communication channel to a remote site; and
d2) means for sending the alarm to the remote site.

22. The system claimed in claim 12, wherein the means for signaling the alarm further comprises:
d1) means for providing a communication channel to a local site; and
d2) means for sending the alarm to the local site.

23. An in vivo camera for employing real-time automatic abnormality detection of in vivo images, comprising:
a) means for forming an examination bundlette of a patient that includes real-time captured in vivo images;
b) means for processing the examination bundlette;
c) means for automatically detecting one or more abnormalities in the examination bundlette based on predetermined criteria for the patient; and
d) means for signaling an alarm provided that the one or more abnormalities in the examination bundlette have been detected.
